(19) United States
(12) Patent Application Publication
(10) Pub. No.: US 2009/0134023 A1
(43) Pub. Date: May 28, 2009

(54) BIOSENSOR

(76) Inventors: Junko Nakayama, Ehime (JP); Yoshifumi Takahara, Ehime (JP); Eriko Yamanishi, Ehime (JP); Yoshihiro Itoh, Ehime (JP)

Correspondence Address: WENDEROTH, LIND & PONACK L.L.P., 1030 15th Street, N.W., Suite 400 East, Washington, DC, 20005-1503, US

(21) Appl. No.: 12/297,672

(22) PCT Filed: Apr. 19, 2007
(86) PCT No.: PCT/JP2007/058526

§ 371 Date: Oct. 20, 2008

(30) Foreign Application Priority Data
Apr. 19, 2006 (JP) 2006-116175

(51) Int. Cl. G01N 27/26; G01N 33/487; C12M 1/00

(52) U.S. Cl. 204/403.14; 204/403.01; 435/287.1

(57) ABSTRACT

In a biosensor of the present invention, BSA is contained in a reagent layer including a reagent which reacts specifically with glucose, and the glucose concentration in whole blood as a sample solution is electrochemically measured. When the sensor response characteristics when whole blood is used as the sample solution are measured, the influence by hematocrit to the sensor response characteristics is reduced according to the additive amount of BSA when the hematocrit value is in the range of 25% and below, while no BSA addition effect is recognized in the sensor response characteristics when the hematocrit value is in the range of 25 to 65%. Thereby, the influences by hematocrit and temperature can be reduced, and thus highly precise measurement can be performed.

### TECHNICAL FIELD

The present invention relates to a biosensor for analyzing a specific component in a sample solution, and more particularly, to a reagent formulation for composing a reagent layer of a biosensor.

### BACKGROUND ART

A biosensor is a sensor which utilizes the molecule identifying abilities of biological materials such as micro-organisms, enzymes, and antibodies to apply the biological materials as molecule recognition elements. To be specific, the biosensor utilizes a reaction which occurs when an immobilized biological material recognizes a target specific component, such as oxygen consumption by respiration of a micro-organism, an enzyme reaction, or luminescence.

Among biosensors, enzyme sensors have been advanced in practical applications, and for example, enzyme sensors for glucose, lactic acid, cholesterol, lactose, uric acid, urea, and amino acid are utilized in medical measurement and food industry. An enzyme sensor reduces an electron acceptor by an electron generated by a reaction between an enzyme and a substrate included in a sample solution as a specimen, and a measurement device electrochemically measures the oxidation-reduction quantity of the electron acceptor, thereby to perform quantitative analysis of the specimen.

FIG. 5 shows an exploded perspective view of a three-electrode-system biosensor as an example of such biosensor.

The biosensor shown in FIG. 5 is fabricated as follows. After an electric conductive layer is formed on an insulating substrate 1 by a sputtering deposition method or a screen printing method, slits are formed using laser or the like to produce a working electrode 2, a counter electrode 3, and a detection electrode 4, and then a reagent layer 5 including an enzyme which reacts with a specific component in a sample solution, and an electron carrier is formed on these electrodes. Further, a spacer 6 having a notch 6a and a cover 8 are bonded together onto the reagent layer 5 and the electrodes 2, 3, and 4, thereby forming a cavity 7 into which the sample solution is supplied. While supply of the sample solution from the cavity 7 into the biosensor is realized by a capillary phenomenon, smooth supply of the sample solution is realized by providing the cover 8 with an air hole 9 for letting the air in the cavity 7 out of the biosensor.

When the sample solution is applied to an inlet of the cavity 7 of thus configured biosensor, the sample solution is supplied from the inlet of the cavity 7 into the cavity 7 by the capillary phenomenon, and when it reaches the position of the reagent layer 5, the specific component in the sample solution reacts with the reagent included in the reagent layer 5. The amount of change in current which occurs due to this reaction is read with an external measurement device which is connected through the leads 10, 11, and 12 of the working electrode 2, the counter electrode 3, and the detection electrode 4, respectively. The read current value is converted into the concentration of the specific component to determine the quantity of the specific component in the sample solution.

Patent Document 1: Japanese Published Patent Application No.

Patent Document 2: Japanese Published Patent Application No.

Patent Document 3: Japanese Published Patent Application No.

### DISCLOSURE OF THE INVENTION

### Problems to be Solved by the Invention

However, the conventional biosensor has a problem that a correct inspection result cannot be obtained due to an influence of hematocrit if the sample solution is blood. Especially an enzyme sensor for glucose is often used for measurement at the time of insulin injection before meal or evaluation for low blood sugar, and there is a possibility of inviting excessive administration of insulin or missing of blood sugar level if the glucose concentration is displayed higher than the actual value due to the influence by hematocrit. Therefore, a highly precise biosensor which is not affected by the influence by hematocrit when the sample solution is blood is desired.

Further, the conventional biosensor has a problem that a correct inspection result cannot be obtained due to an influence by ambient temperature. Although the inspection result is corrected using a temperature control device or the like to resolve this problem, wrong correction might be performed if the temperature control device cannot respond to a rapid temperature change and falsely recognizes the temperature, and a correct inspection result cannot be obtained. Accordingly, a biosensor which is hardly affected by the influence by ambient temperature is demanded.

The present invention is made to solve the above-described problems and has for its object to provide a highly precise biosensor which can avoid the influence by hematocrit and the influence by ambient temperature.

### Measures to Solve the Problems

In order to solve the above-described problems, according to claim 1 of the present invention, there is provided a biosensor for measuring the concentration of a specific component in a sample solution, wherein a solubilized protein is contained in a reagent layer including a reagent which reacts specifically with the specific component in the sample solution.

According to claim 2 of the present invention, in the biosensor defined in claim 1, the solubilized protein is bovine serum albumin, egg albumin, gelatin, or collagen.

According to claim 3 of the present invention, in the biosensor defined in claim 1, the amount of the solubilized protein contained is within a range of 0.0004 to 0.008 mg per enzyme 1 U.

According to claim 4 of the present invention, in the biosensor defined in claim 1, the amount of the solubilized protein contained is within a range of 0.0007 to 0.014 mg per one sensor.

According to claim 5 of the present invention, in the biosensor defined in claim 3, the amount of the solubilized protein contained is within a range of 0.0035 to 0.004 mg per enzyme 1 U.

According to claim 6 of the present invention, in the biosensor defined in claim 4, the amount of the solubilized protein contained is 0.007 ng per one sensor.

According to claim 7 of the present invention, in the biosensor defined in claim 1, the concentration of the specific component is measured using electrodes including at least a working electrode and a counter electrode, which electrodes are provided on an insulating substrate.

According to claim 8 of the present invention, in the biosensor defined in claim 7, the reagent layer includes at least an enzyme and an electron carrier, and the reagent layer is formed on the electrodes, or formed so that the electrodes are disposed in a diffusion area in which the reagent of the reagent layer is dissolved in the sample solution and diffused.

According to claim 9 of the present invention, in the biosensor defined in claim 8, the enzyme is glucose dehydrogenase having flavin adenine dinucleotide as a coenzyme, or glucose dehydrogenase having pyrrolo-quinoline quinone as a coenzyme.

### EFFECTS OF THE INVENTION

According to the present invention, in a biosensor for measuring the concentration of a specific component in a sample solution, a solubilized protein is contained in a reagent layer including a reagent which reacts specifically with the specific component in the sample solution, thereby realizing a highly precise biosensor which reduces the influence by hematocrit and the influence by temperature.

### BRIEF DESCRIPTION OF THE DRAWINGS

FIG. 1 is a diagram illustrating the influence by hematocrit to sensor response characteristics in the case where BSA is added to a reagent layer in a biosensor according to a first embodiment of the present invention.

FIG. 2 is a diagram illustrating the influence by temperature to sensor response characteristics in the case where BSA is added to the reagent layer in the biosensor according to the first embodiment.

FIG. 3 is a diagram illustrating the influence by hematocrit to sensor response characteristics in the case where BSA is added to a reagent layer in a biosensor according to a second embodiment of the present invention.

FIG. 4 is a diagram illustrating the influence by temperature to sensor response characteristics in the case where BSA is added to the reagent layer in the biosensor according to the second embodiment.

FIG. 5 is an exploded perspective view of a conventional three-electrode-system biosensor.

### DESCRIPTION OF REFERENCE NUMERALS

1 . . . substrate—
2 . . . working electrode
3 . . . counter electrode
4 . . . detection electrode
5 . . . reagent layer
6 . . . spacer
6a . . . notch
7 . . . cavity
8 . . . cover
9 . . . air hole
10,11,12 . . . leads

### BEST MODE TO PERFORM THE INVENTION

Hereinafter, preferred embodiments of the present invention will be described with reference to the drawings. In the respective embodiments of the present invention described hereinafter, an enzyme sensor which adopts an enzyme as a molecular identification element that reacts specifically with a specific component in a sample solution will be exemplified.

### Embodiment 1

A biosensor according to a first embodiment of the present invention will be described.

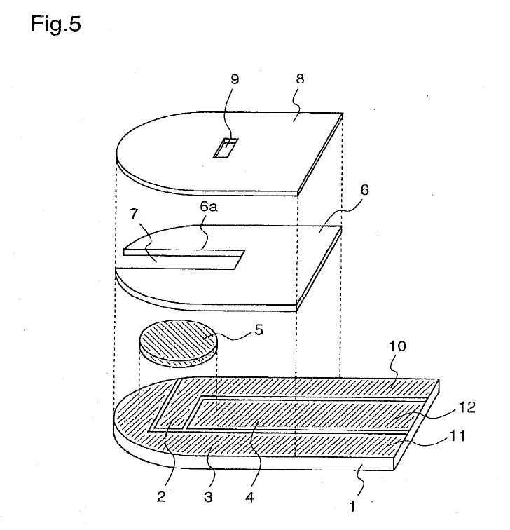

The biosensor of this first embodiment is characterized by that a solubilized protein is added to the reagent layer 5 of the biosensor shown in FIG. 5. In this first embodiment, glucose dehydrogenase having flavin adenine dinucleotide as a coenzyme (hereinafter referred to as FAD-GDH) is used as an enzyme. Further, bovine serum albumin (hereinafter referred to as BSA) is used as a solubilized protein.

Hereinafter, the function and effect of this first embodiment will be described.

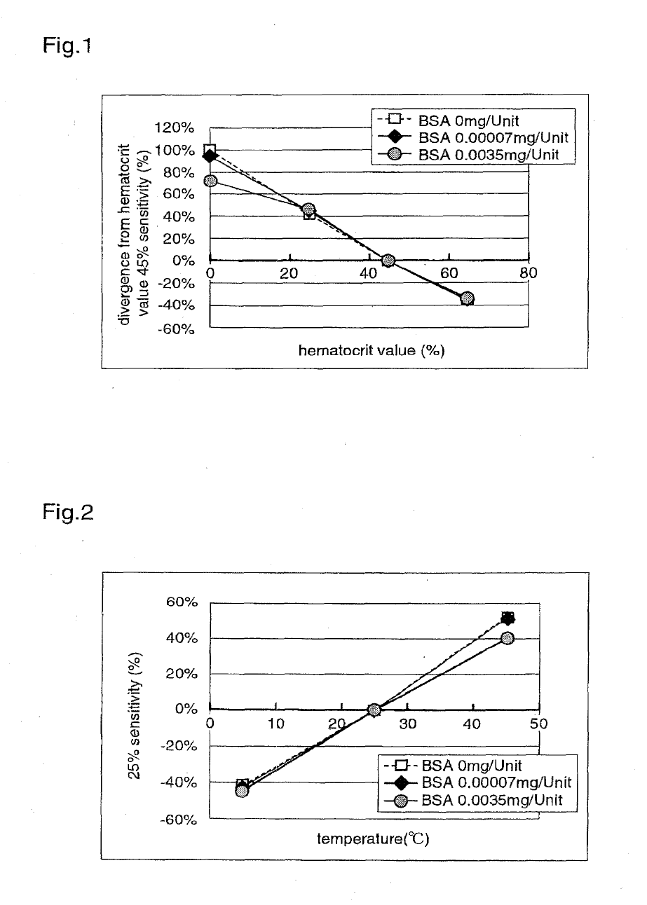

FIG. 1 is a diagram illustrating the influence by hematocrit to sensor response characteristics when BSA is added to the reagent layer 5 in the biosensor of the first embodiment. In FIG. 1, the ordinate shows the divergence from the sensor sensitivity when the hematocrit value is 45%, and the abscissa shows the hematocrit value.

Whole blood having a glucose concentration adjusted to 350 mg/dL is used as a sample solution. Further, the enzyme amount of FAD-GDH is 2 U (unit) per one sensor.

The sensor response value is measured with the concentration of BSA added to the reagent layer 5 which constitutes the biosensor of the first embodiment being varied from 0 to 0.0035 mg per enzyme 1 U, in other words, from 0 to 0.007 mg per one sensor in the reagent solution.

As the result of measurement, in the concentration range where the hematocrit value is 25 to 65%, no change is found in the sensor response value even when BSA is added. On the other hand, in the low concentration range where the hematocrit value is 25% and below, the influence by hematocrit to the sensor response characteristics is reduced according to the additive amount of BSA.

In this way, when FAD-GDH and BSA are contained in the reagent layer 5, BSA addition effect is recognized in the low concentration range where the hematocrit value is 0 to 25%, while no BSA addition effect is recognized in the concentration range where the hematocrit value is 25% and above.

FIG. 2 is a diagram illustrating the influence by temperature to the sensor response characteristics when BSA is added to the reagent layer 5 in the biosensor of the first embodiment. In FIG. 2, the ordinate shows the divergence from the sensitivity at temperature 25° C., and the abscissa shows the temperature.

Whole blood having a glucose concentration adjusted to 350 mg/dL is used as a sample solution. Further, the enzyme amount of FAD-GDH is 2 U per one sensor.

The sensor response characteristics are measured with the concentration of BSA added to the reagent layer 5 which constitutes the biosensor of the first embodiment being varied from 0 to 0.0035 mg per enzyme 1 U, in other words, from 0 to 0.007 mg per one sensor in the reagent solution.

As the result of measurement, in the temperature range of 5 to 25° C., no change is found in the sensor response value even when BSA is added. On the other hand, in the high temperature range of 25° C. and above, the influence by temperature to the sensor response characteristics is reduced according to the additive amount of BSA.

In this way, when FAD-GDH and BSA are contained in the reagent layer 5, BSA addition effect is recognized in the temperature range higher than 25° C., while no BSA addition effect is recognized in the temperature range of 25° C. and below.

Consequently, it can be determined that the characteristics of both the hematocrit and the temperature can be enhanced when BSA is included in the reagent layer 5 by 0.0035 mg per enzyme 1 U or by 0.007 mg per one sensor.

According to the biosensor of this first embodiment, since a large amount of BSA is contained in the reagent layer including the enzyme FAD-GDH, the influences by hematocrit and temperature to the sensor response characteristics can be improved.

### Embodiment 2

A biosensor according to a second embodiment of the present invention will be described.

The biosensor of this second embodiment is characterized by that a solubilized protein is added to the reagent layer 5 of the biosensor shown in FIG. 5. In this second embodiment, glucose dehydrogenase having pyrrolo-quinoline quinone as a coenzyme (hereinafter referred to as PQQ-GDH) is used as an enzyme.

Further, bovine serum albumin (hereinafter referred to as BSA) is used as a solubilized protein.

Hereinafter, the function and effect of this second embodiment will be described.

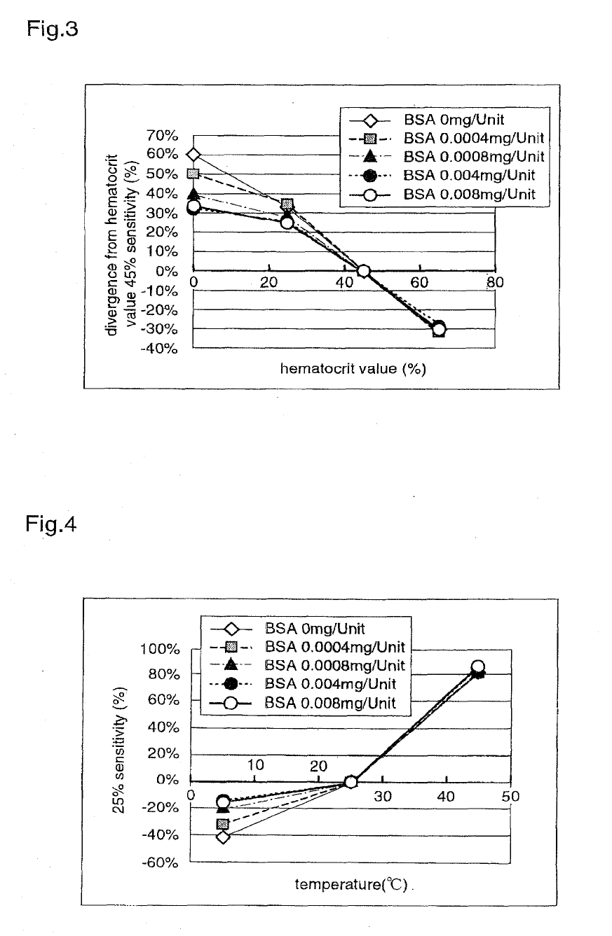

FIG. 3 is a diagram illustrating the influence by hematocrit to sensor response characteristics when BSA is added to the reagent layer 5 in the biosensor of the second embodiment. In FIG. 3, the ordinate shows the divergence from the sensor sensitivity when the hematocrit value is 45%, and the abscissa shows the hematocrit value.

Whole blood having a glucose concentration adjusted to 350 mg/dL is used as a sample solution. Further, the enzyme amount of PQQ-GDH is 1.7 U per one sensor.

The sensor response value is measured with the concentration of BSA added into the reagent layer 5 which constitutes the biosensor of the second embodiment being varied from 0 to 0.008 mg per enzyme 1 U, in other words, from 0 to 0.014 mg per one sensor in the reagent solution.

As the result of the measurement, no BSA addition effect to the sensor response value is recognized in the region where the hematocrit value is 25 to 65%. On the other hand, in the low concentration range where the hematocrit value is 25% and below, the influence by hematocrit to the sensor response characteristics is reduced according to the additive amount of BSA.

In this way, when PQQ-GDH and BSA are contained in the reagent layer 5, BSA addition effect is recognized in the low concentration range where the hematocrit value is 0 to 25%, while no BSA addition effect is recognized in the concentration range where the hematocrit value is 25% and above.

FIG. 4 is a diagram illustrating the influence by temperature to the sensor response characteristics when BSA is added to the reagent layer 5 in the biosensor of the second embodiment. In FIG. 2, the ordinate shows the divergence from the sensitivity at temperature 25° C., and the abscissa shows the temperature.

Blood plasma having a glucose concentration adjusted to 350 mg/dL is used as a sample solution. The enzyme amount of PQQ-GDH is 1.7 U per one sensor.

The sensor response characteristics are measured with the concentration of BSA added to the reagent layer 5 which constitutes the biosensor of the second embodiment being varied from 0 to 0.008 mg per enzyme 1 U, in other words, from 0 to 0.014 mg per one sensor in the reagent solution.

As the result of measurement, in the temperature range of 25 to 45° C., no change is found in the sensor response value even when BSA is added. On the other hand, in the low temperature range of 25° C. and below, the influence by temperature to the sensor response characteristics is reduced according to the additive amount of BSA.

In this way, when PQQ-GDH and BSA are contained in the reagent layer 5, BSA addition effect is recognized when the temperature is lower than 25° C., while no BSA addition effect is recognized when the temperature is 25° C. or above.

Consequently, it can be determined that the characteristics of both the hematocrit and the temperature can be enhanced when BSA is included in the reagent layer 5 by 0.0004 to 0.008 mg per enzyme 1 U or by 0.0007 to 0.014 mg per one sensor. Further, since the effect is not changed when the BSA is added by more than 0.004 mg per enzyme 1 U or by more than 0.007 mg per one sensor, it can be determined that the optimum value of BSA is 0.004 mg per enzyme 1 U or 0.007 mg per one sensor.

According to the biosensor of this second embodiment, since a large amount of BSA is contained in the reagent layer including the enzyme PQQ-GDH, the influences by hematocrit and temperature to the sensor response characteristics can be improved.

While in the first and second embodiments FAD-GDH and PQQ-GDH are adopted as the enzymes in the reagent layer 5, there may be adopted other enzymes used for clinical inspection, such as cholesterol oxidase, cholesterol esterase, cholesterol dehydrogenase, lipoprotein lipase, catalase, peroxidase, lactate oxidase, lactate dehydrogenase, urease, uricase, glucose oxidase, glucose dehydrogenase, hexokinase, ascorbic acid oxidase, ascorbic acid dehydrogenase, diaphorase, and the like.

While in the first and second embodiments BSA is adopted as the solubilized protein, the same effects can be achieved also when egg albumin, gelatin, collagen, or the like is adopted. It can be determined that the optimum value of the additive amount of the solubilized protein is 0.004 mg per enzyme 1 U or 0.007 mg per one sensor.

While in the first and second embodiments the three-electrode-system biosensor is described, a two-electrode-system biosensor may be used.

### APPLICABILITY IN INDUSTRY

A biosensor of the present invention can be utilized as a highly precise enzyme sensor which can reduce the influence by hematocrit as well as the influence by temperature.

1. A biosensor for measuring the concentration of a specific component in a sample solution, wherein a solubilized protein is contained in a reagent layer including a reagent which reacts specifically with the specific component in the sample solution.

2. A biosensor as defined in claim 1, wherein the solubilized protein is bovine serum albumin, egg albumin, gelatin, or collagen.

3. A biosensor as defined in claim 1, wherein the amount of the solubilized protein contained is within a range of 0.0004 to 0.008 mg per enzyme 1 U.

4. A biosensor as defined in claim 1, wherein the amount of the solubilized protein contained is within a range of 0.0007 to 0.014 mg per one sensor.

5. A biosensor as defined in claim 3, wherein the amount of the solubilized protein contained is within a range of 0.0035 to 0.004 mg per enzyme 1 U.

6. A biosensor as defined in claim 4, wherein the amount of the solubilized protein contained is 0.007 mg per one sensor.

7. A biosensor as defined in claim 1, wherein the concentration of the specific component is measured using electrodes including at least a working electrode and a counter electrode, which electrodes are provided on an insulating substrate.

8. A biosensor as defined in claim 7, wherein the reagent layer includes at least an enzyme and an electron carrier, and said reagent layer is formed on the electrodes, or formed so that the electrodes are disposed in a diffusion area in which the reagent of the reagent layer is dissolved in the sample solution and diffused.

9. A biosensor as defined in claim 8, wherein the enzyme is glucose dehydrogenase having flavin adenine dinucleotide as a coenzyme, or glucose dehydrogenase having pyrrolo-quinoline quinone as a coenzyme.
